SEQUENCE LISTING (3 sequences)

SEQ ID NO: 1 - DNA, 20 bp, homo sapiens
  misc_feature (1)..(3): FAM Fluorescein
  misc_feature (13)..(15): Quencher
  misc_feature (27)..(33): Quencher
agcacgtgggagggcgatcg

SEQ ID NO: 2 - DNA, 26 bp, homo sapiens
atgttcagrtggatgagrttctcwga

SEQ ID NO: 3 - DNA, 21 bp, homo sapiens
tcgacgccatcttcattcaca

(19) United States
(12) Patent Application Publication
(10) Pub. No.: US 2023/0145615 A1
(43) Pub. Date: May 11, 2023

(54) TREATING OR PREVENTING TRAVELERS DIARRHEA

(71) Applicant: SCALED MICROBIOMICS, LLC, Hagerstown, MD (US)

(72) Inventor: Julius G. GOEPP, Hagerstown, MD (US)

(21) Appl. No.: 17/918,417

(22) PCT Filed: Apr. 14, 2021
(86) PCT No.: PCT/US2021/027264

§ 371 Date: Oct. 12, 2022

Related U.S. Application Data
(60) Provisional application No. 63/009,509, filed on Apr. 14, 2020.

(51) Int. Cl. C07K 16/12; C07K 16/10; A61P 31/14; A61P 31/04

(52) U.S. Cl. CPC C07K 16/1232; C07K 16/10; A61P 31/14; A61P 31/04; A61K 39/00

(57) ABSTRACT

In one aspect, a formulation comprising (a) an antibody that specifically binds an enterotoxigenic Escherichia coli (ETEC) or a molecule produced by an ETEC and (b) an antibody that specifically binds a norovirus (NV) or epitope thereof, is provided. In another aspect, a method of treating or preventing Travelers' diarrhea using the formulation is provided.

### CROSS-REFERENCE TO RELATED APPLICATIONS

This application claims priority to PCT International Application No. PCT/US2021/027264, titled “Treating or Preventing Travelers Diarrhea,” filed Apr. 14, 2021 which claims priority to U.S. Provisional Application Ser. No. 63/009,509 filed on Apr. 14, 2020, each of which is incorporated herein, in its entirety, by this reference.

### SEQUENCE LISTING

This named application incorporates by reference the Sequence Listing contained in an ASCII text file “369378 00022 SeqList.text” submitted via EFS-Web. The text file was created on Oct. 12, 2022, and is 929 bytes in size.

### BACKGROUND OF THE INVENTION

Travelers' diarrhea (TD) affects 40-60% of travelers from industrialized to less-developed nations, including about 9.5-14.9 million US travelers and 7.6-12.5 million European travelers annually, resulting into financial losses to destination countries and the travel industry. Lost productivity due to TD in the United States is estimated to be about $645 million annually. Current TD prevention is inadequate. Antibiotics, often carried by travelers to use as prophylaxis against TD, are both ineffective against HuNV, and generally contraindicated for prophylaxis because of their potential damage to the host microbiome and the risk of selecting antibiotic-resistant organisms that can remain in the environment and pose threats to others. Because of the lack of safe, effective, and licensed vaccines against ETEC and HuNV, there exists a long-felt need for rapid and effective prophylaxis against acquisition of infection by either of these microorganisms alone or in combination.

### SUMMARY OF THE INVENTION

In one aspect, an antibody that specifically binds an enterotoxigenic Escherichia coli (ETEC) or a molecule produced by an ETEC, is provided. In another aspect, a formulation that includes one or more mixtures of polyclonal antibodies comprising an antibody that specifically binds an enterotoxigenic Escherichia coli (ETEC) or a molecule produced by an ETEC, is provided. In some embodiments, the antibody is an IgY antibody. In some embodiments, the molecule produced by an ETEC is an adhesin. In some other embodiments, the antibody binds a multiepitope fusion antigen (MEFA). In still other embodiments, the antibody inhibits ETEC adhesion to Caco-2 cells and Vero cells.

In another aspect, an antibody that specifically binds a norovirus (NV) or epitope thereof, is provided. In still another aspect, a polyclonal mixture of antibodies comprising an antibody that specifically binds a norovirus (NV) or epitope thereof, is provided. In some embodiments, the antibody is an IgY antibody. In some embodiments, the NV is NV GII.4 or NV GI.1. In some other embodiments, the antibody blocks binding of NV to histo-blood group antigen (HBGA). In yet other embodiments, the antibody is produced against a specific NV strain, for non-limiting example, GII.4/CHDC2094/1974/US, and is effective against at least one additional NV strain, for non-limiting example, GII.4 Sydney [P16].

In still another aspect, a mixture of antibodies is provided. The mixture contains (a) an antibody that specifically binds an enterotoxigenic Escherichia coli (ETEC) or a molecule produced by an ETEC and (b) an antibody that specifically binds a norovirus (NV) or epitope thereof In some embodiments of any one of the mixtures of antibodies described herein, the mixture is multivalent.

In another aspect, a formulation for treating or preventing Travelers' diarrhea (TD) is provided, the formulation comprising a mixture of (a) an antibody that specifically binds an enterotoxigenic Escherichia coli (ETEC) or a molecule produced by an ETEC and (b) an antibody that specifically binds a norovirus (NV) or epitope thereof. The mixture of antibodies can be any one of the mixtures described herein. In some embodiments, the formulation is formulated for oral administration.

In yet another aspect, a method of treating or preventing Travelers' diarrhea in a subject in need thereof is provided. The method includes the step of administering to the subject a formulation that contains (a) an antibody that specifically binds an enterotoxigenic Escherichia coli (ETEC) or a molecule produced by an ETEC and (b) an antibody that specifically binds a norovirus (NV) or epitope thereof, in advance of a potential exposure to either microorganism. Alternatively or in addition, the formulation is administered following exposure, with the intent of mitigating symptoms. In some embodiments, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts IgY Titers for anti-ETEC Adhesin Tip MEFA IgY and anti-HuNV GII.4 CHDC2094/1974 IgY. The graph demonstrates successful and sustained production of both anti-ETEC adhesin-tip MEFA IgY and anti-GII.4 CHDC2094/1974 over the course of 24 weeks following initial immunizations by intramuscular injection of the hens. This level of antibody production remains stable at nine months following initial immunization.

FIG. 2 depicts SDS-PAGE Analysis of anti-ETEC Adhesin Tip MEFA IgY and anti-HuNV GII.4 CHDC2094/1974 IgY. The results demonstrate the characteristic SDS-PAGE bands of IgY for both anti-ETEC adhesin-tip MEFA and anti-HuNV GII.4 CHDC2094/1974 IgY at 65 and 27 kDa (heavy and light chains respectively).

FIG. 3 demonstrates the specific binding of anti-ETEC adhesin-tip MEFA IgY to each of the nine adhesins represented on the MEFA. This is an essential demonstration of epitope-specific binding, which is different from binding of IgY to the intact MEFA, and indicates likelihood that the anti-MEFA IgY will in fact interact strongly with ETEC strains bearing one or more of those adhesin epitopes. The reactivity of unimmunized IgY with each of the nine adhesins is shown to be zero, indicating the lack of intrinsic anti-adhesin antibodies in unimmunized hens.

FIG. 4 depicts graphs demonstrating that anti-ETEC adhesin-tip MEFA IgY prevents adhesion of MEFA design strains to Caco2. The results illustrate significant inhibition of adhesion by ETEC strains used in development of the adhesin-tip MEFA to Caco2 cells in culture, by comparison with unimmunized IgY.

FIG. 5 depicts results demonstrating that anti-ETEC adhesin-yip MEFA IgY prevents adhesion of outbreak-associated ETEC strains to vero. Specifically, the results demonstrate significant inhibition of adhesion of seven outbreak-associated ETEC strains, including one (31-10) bearing CFA/III, not found on the MEFA, and hence an indication of a degree of cross-reactivity to non-MEFA CFAs. FIG. 5 also shows a single outbreak-associated ETEC strain (MP215-1) bearing CFA/III, that showed no significant inhibition of adhesion to mammalian cells. In practice, CFA/III is found on only a small fraction of disease-producing ETEC strains.

FIG. 6 demonstrates the lack of impact of anti-ETEC adhesin-tip MEFA IgY on growth of several ETEC and non-ETEC strains of E. coli. In all cases the antibiotic ciprofloaxacin was shown to be bactericidal, while no change in growth or bacterial survival was seen in IgY-treated organisms.

FIG. 7 demonstrates significant inhibition by anti-HuNV GII.4 CHDC2094/1974 IgY to HBGA antigens in a cell-free system, at dilutions from “neat” (10 mg/mL IgY protein) up to 1:1,000, indicating that the IgY blocks those aspects of the VLP involved with binding to HBGA, the requisite first step in establishing HuNV infection.

FIG. 8 demonstrates significant reduction by anti-HuNV GII.4 CHDC2094/1974 IgY of viral replication of the more-recent HuNV GII.4 [P16] Sydney strain in a human intestinal enteroid model.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rigger et al. (eds.), Springer Verlag (1991); and Hale & Marham, The Harper Collins Dictionary of Biology (1991). As used herein, the following terms have the meanings ascribed to them below, unless specified otherwise.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

By “ameliorate” is meant decrease, suppress, attenuate, diminish, arrest, or stabilize the development or progression of a disease.

The term “antibody,” as used herein, refers to an immunoglobulin molecule that specifically binds with an antigen. Antibodies can be intact immunoglobulins derived from natural sources or from recombinant sources, and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, single-domain antibodies such as sdAb (either VL or VH), such as camelid antibodies (Riechmann, 1999, J. Immunol. Meth. 231:25-38), camelid VHH domains, composed of either a VL or a VH domain that exhibit sufficient affinity for the target, and multispecific antibodies formed from antibody fragments such as a bivalent fragment comprising two Fab fragments linked by a disulfide bridge at the hinge region, and an isolated complementarity-determining region (CDR) or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger & Hudson, 2005, Nature Biotech. 23:1126-1136). Antigen binding fragments can also be grafted into scaffolds based on polypeptides such as a fibronectin type III (Fn3) (U.S. Pat. No. 6,703,199, which describes fibronectin polypeptide minibodies). The antibody fragment also includes a human antibody or a humanized antibody or a portion of a human antibody or a humanized antibody.

In one aspect of the present invention, the non-human antibody is an avian antibody, immunoglobulin Y (IgY). As will be understood by those skilled in the art, production of IgY necessarily entails changes in the host hen's immune tissues that permit extraction of genetic material for introduction into single celled expression systems, including but not limited to yeast cells, Chinese hamster ovary (CHO) cells, and human hybridoma cells. Antibodies produced in such systems are known as “engineered antibodies” or “antibody fragments” or “nanobodies.” It is therefore possible to screen native polyclonal IgY for antibodies with the desired effects on a microorganism-produced molecule, identify those with the strongest desirable characteristics, and proceed to produce engineered antibodies with identical or superior characteristics to those in the native polyclonal mixture produced by the hen.

In some other aspects, the non-human antibody is a mammalian antibody, of the immunoglobulin class G (IgG), A (IgA and secretory IgA), or M (IgM). In some embodiments, the antibody or fragment thereof is a monomeric IgA, such as the IgA described in Virdi et al. Nat. Biotechnol. 2019 May;37(5):527-530. In some embodiments, the antibody or fragment thereof is a VHH, such as for example, llama-derived single chain antibody fragments (VHH) as described in Garaicoechea et al., 2015 PloS ONE 10(8):e0133665.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “animal” refers to living multi-cellular vertebrate organisms, a category that includes, for example, mammals and birds. The term “mammal” includes both human and non-human mammals.

As used herein, “immunoglobulin” refers to a polypeptide ligand comprising at least a light chain or heavy chain immunoglobulin variable region which specifically binds an epitope of a protein or a fragment of a protein. Immunoglobulins can include a heavy chain and a light chain, each of which has a variable region, termed the variable heavy (VH) region and the variable light (VL) region. Together, the VH region and the VL region are responsible for binding the antigen recognized by the immunoglobulin. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric immunoglobulins (for example, humanized murine immunoglobulins), heteroconjugate immunoglobulins (such as, bispecific immunoglobulins), and immunoglobulins produced by genetically-modified bacteria or yeast under defined conditions. See also, Pierce Catalog and Handbook, 1994-1995 (Pierce Chemical Co., Rockford, Ill.); Kuby, Immunology, 3rd Ed., W.H. Freeman & Co., New York, 1997.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

By “disease” is meant any condition or disorder that damages or interferes with the normal function of a cell, tissue, or organ, system or entire organism.

The terms “effective amount” or “therapeutically effective amount” are used interchangeably herein, and refer to an amount of a compound, antibody or antigen-binding fragment thereof, formulation, material, or composition, as described herein effective to achieve a particular biological result. Such results may include, but are not limited to, the treatment of a disease or condition as determined by any means suitable in the art.

As used herein, “epitope” means a molecular structure that is recognized by the immune system and leads to the production of specific immunoglobulins directed against the epitope.

As used herein, “immunoglobulin Y” (“IgY”) is a type of immunoglobulin which is the major immunoglobulin in bird, reptile, and lungfish blood. It is also found in high concentrations in chicken egg yolk. As with the other immunoglobulins, IgY is a class of proteins which are formed by the immune system in reaction to certain foreign substances, and specifically recognize them. IgY is composed of two light and two heavy chains. Structurally, these two types of immunoglobulin differ primarily in the heavy chains, which in IgY have a molecular mass of about 65,100 atomic mass units (amu). The light chains in IgY have a molar mass of about 18,700 amu. The molar mass of IgY thus amounts to about 167,000 amu.

An “individual”, “patient” or “subject”, as these terms are used interchangeably herein, includes a member of any animal species including, but are not limited to, birds, humans and other primates, and other mammals including commercially relevant mammals such as cattle, pigs, horses, sheep, cats, and dogs. Preferably, the subject is a human. As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “therapeutically effective amount” or “effective amount” or “therapeutically effective dose” is that amount or dose sufficient to inhibit or prevent onset or advancement, to treat outward symptoms, or to cause regression, of a disease. The therapeutically effective amount or dose also can be considered as that amount or dose capable of relieving symptoms caused by the disease. Thus, a therapeutically effective amount or dose of an anti-fungal agent is that amount or dose sufficient to achieve a stated therapeutic effect. The therapeutically effective amount may vary depending the subject and disease condition being treated, e.g., the weight and age of the subject, the severity of the disease condition, the manner of administration and the like, which can readily be determined by one of ordinary skill in the art.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention generally relates to reducing the risks and manifestations of acute gastroenteritis (AGE) among travelers, also called Travelers' Diarrhea (TD). More particularly, the present invention relates to means of preventing AGE caused by enterotoxigenic Escherichia coli (ETEC) and Human Norovirus (HuNV), the primary bacterial and viral microorganisms responsible for AGE in endemic settings and in travelers to areas where these pathogens are endemic.

In some embodiments, the present invention relates to oral, broad-spectrum formulations of anti-ETEC and anti-NV IgY that provide immediate passive immunity to multiple strains of both organisms. To date, no single therapeutic or prophylactic meets all of these conditions, especially the requirement for coverage of both ETEC and HuNV, together overwhelmingly the leading causal agents of TD. Further, such a combination prophylactic must be effective against the most-prevalent strains of each microorganism, the numbers of which exceed ten variants of both ETEC and HuNV. In certain embodiments, the present invention provides prophylactic formulations that are also fungible, in that the formulation can be readily modified to include emerging strains of either microorganism, and indeed can be altered to include other diarrheal pathogens as required.

The present invention provides a formulation for reducing the risk of, or treating, AGE associated with travel to areas where both microorganisms are endemic, the formulation consisting of a therapeutically effective amount of at least one polyclonal IgY antibody specific to multiple strains of ETEC and at least one polyclonal IgY antibody specific to multiple genotypes and genotype variants of HuNV. In some embodiments, the at least one polyclonal IgY is raised against a multiepitope fusion antigen (MEFA) that is a protein construct consisting of an immunogenic backbone to which are covalently bound peptides representing epitopes of ETEC adhesin tips that vary among pathogenic ETEC strains.

Disease production by ETEC requires a) adhesion to ileal mucosa, b) colonization, and c) toxin production. ETEC strains produce at least 23 distinct colonization factor adhesins (CFAs); after colonization, ETEC expresses heat-labile (LT) and heat-stable (ST) toxins that elevate intracellular cyclic adenosine monophosphate (cAMP) and cyclic guanosine monophosphate (cGMP) intracellular messengers that disrupt fluid homeostasis and ultimately induce diarrhea. ETEC strains producing one or more CFAs plus either toxin are pathogenic. A total of nine distinct CFAs are found in the 23 ETEC strains known to produce diarrhea: CFA/I, CFA/II (coli surface antigens (CS) CS1, CS2, CS3), CFA/IV (CS4, CS5, CS6), the Longus pilus (CS21) and outer membrane protein adhesin (EtpA). The toxins produced by ETEC organisms represent additional potential antigenic targets.

Variations in those epitopes between multiple ETEC strains are responsible in part for the challenges in developing a generally-applicable ETEC vaccine; such variations are troublesome for any prophylactic method aimed at the ETEC organisms. The MEFA used in production of anti-ETEC IgY for this invention was developed as a human vaccine candidate but has not been commercialized in a human vaccine; here it is described as the primary immunogen for production of IgY to be used as passive, orally-administered immunoprophylaxis for human use, an application which is entirely novel.

In some embodiments, the one or more antibodies provided by the present invention include one or more polyclonal IgY antibodies raised against a virus-like particle (VLP) comprised of repeating units of the HuNV capsid protein VP1, which is specified by open reading frame 2 (ORF2) on the HuNV genome.

A similar degree of diversity is now recognized in HuNV strains. The variation is accounted for by small alterations in the amino acid sequences of the VP1 capsid proteins used by HuNV to adhere to histo-blood group antigens (HBGA) on human intestinal cells. VP1 proteins vary in peptide sequences both between genogroups and genotypes of HuNV, and also within genotypes that were previously considered invariant. This variability in HuNV capsid proteins are responsible in part for challenges in developing a HuNV vaccine, and is troublesome for any prophylactic method aimed at the HuNV organisms. As such, the formation of the present invention may be altered and/or adjusted in order to address and account for this variability.

The present invention provides multivalent formulations for preventing TD caused by any of multiple ETEC or any of multiple HuNV strains. The use of IgY as described herein permits the immediate targeting of both ETEC and HuNV by ingestion of a therapeutically effective amount of IgY, because coverage will be effective shortly after a first oral dose, and for as long as the oral mixture is used continuously. This is in contradistinction to any vaccine, which, once available, will require a waiting period of several weeks prior to expected exposure to either pathogen, and often a booster vaccine as well. This poses a disincentive, especially to short-term travelers who may not wish to undergo multiple immunizations ahead of a relatively brief trip. Because travelers cannot predict which microorganism they may encounter, this invention provides the further advantage of covering, a priori, organisms that between them account for more than 80% of TD cases.

The formulations of the present invention further provide a broad spectrum of protection. Because of the variations in adhesins (ETEC) and VP1 (HuNV), no single antibody, even directed at either ETEC or HuNV, is likely to produce coverage broad enough to account for variants that will be encountered by travelers in practice. The polyclonal nature of IgY as extracted from eggs has been shown by our laboratory to cover even some strains of both ETEC and HuNV not included in the immunizing material. For example, although the ETEC adhesin-tip MEFA lacks any epitope representing colonization factor antigen (CFA)/III, the anti-ETEC adhesin-tip MEFA IgY blocks coverage of some CFA/III-bearing ETEC variants. Similarly, IgY produced by immunization of hens with VLP of HuNV GII.4/CHDC2094/1974/US is effective at neutralizing replication of HuNV GII.4 Sydney [P16], a variant that emerged more than 30 years after the immunizing strain, and that has known sequence heterology in the VP1 protein with the immunizing strain.

### Methods of Producing IgY

Advantages of use of IgY in general include its high abundance, at roughly 100 mg IgY/egg yolk, making possible production of kilogram (kg) quantities of IgY from even a relatively small commercial laying flock, its ease of extraction by simple physicochemical means, its inability to fix mammalian complement, and its lack of responsiveness to mammalian epitopes. Continuous production has been demonstrated in our laying hens at high levels (greater than 1:131,072, or 1:2¹⁷) for more than nine months after the primary immunization. Further advantages of IgY as a passive immunoprophylactic include its high stability at pH between 4 and 9, and at temperatures up to 60 degrees Celsius. These features are essential for ease of packaging and transportation of the finished product.

In another aspect of this invention, hens may be immunized with multiple antigens simultaneously. Hens can be immunized with up to 20 distinct antigens without loss of antibody production; this is another advantageous feature of this invention. Each targeted IgY can be produced separately by immunization of different groups of hens, permitting titration of the amount of each IgY as required. Alternatively, both antigens may be administered to laying hens together, resulting in production of a mixture of IgYs and simplifying production. The introduction of enabling technologies such as aerosolized immunizations, viral vector immunizations, and in ovo immunizations are all examples of production-related advantages over other means of producing large amounts of polyclonal antibodies rapidly.

In some embodiments, the present invention relates to methods of producing IgY using for example specifically-immunized laying hens. The methods include first identifying the molecular structure of the antigen responsible for pathogenic effects of the target microorganisms. The molecular structure may include one or more adhesin tips that vary between ETEC strains and/or one or more VP1 proteins that vary between HuNV strains.

Embodiments of the methods further include constructing the antigen analogous to the one or more molecular entities of interest using any of several means known to those skilled in the art. For example, the one or more antigens may be constructing using chemical synthesis, expression by recombinant DNA technology in an appropriate bacterial expression system, and others.

Embodiments of the methods further include mixing the produced antigen with an appropriate avian adjuvant, for illustrative example Montanide ISO 70 VG, to boost immune responses in the laying hen.

Embodiments of the methods further include administering the one or more produced antigens to a host organism in order to generate antibodies to the one or more antigens. In some embodiments, the host organism is an avian. Antigens can be administered to the avian host by any of a number of means recognized by those skilled in the art; these include intramuscular inject at one or more time points, aerosolization of antigen in contact with the avian hosts, use of viral vector technology, which permits a single immunization after which the antigen is continuously produced by the host, and in ovo vaccination of the laying hen as an embryo. Each of these methods has advantages, however, in all cases, once the antigen has come into contact with the laying hen's immune system, she begins to produce IgY specific to the target antigen within a few weeks of initial exposure.

After evidence that specific IgY is being produced and deposited in eggs of immunized hens, embodiments of the methods include extracting the IgY by any of a number of published methods to separate it from most other yolk proteins, and subsequently prepared for incorporation at a therapeutically effective amount in an oral capsule, tablet, suspension or other standard drug delivery system.

### Formulations/Methods of Use/Administration/Dosage

In some embodiments, a therapeutically-effective amount of anti-ETEC IgY is encapsulated with a therapeutic amount of anti-HuNV IgY, together with required excipients, in a capsule form resistant to degradation by stomach acid and small intestinal proteases. In this embodiment, the therapeutically effective amount of each IgY is measured in International Units (IU), and the amount of each IgY in IU may differ between the anti-ETEC and anti-HuNV IgY, with the amounts determined by results of animal and human dosing studies.

In some embodiments, the antigens used to produce the IgY are mixed together and administered simultaneously to a group of laying hens, the resulting IgY containing high activity against each target antigen. In this embodiment, encapsulation and delivery of the formulation is otherwise identical to the preferred embodiment.

In some embodiments, the therapeutically effective amounts of anti-ETEC and anti-HuNV IgY are microencapsulated by a protective coating and packaged in an acid-resistant capsule. In this embodiment, the microcapsules are designed in a fashion to deliver the IgY to the upper ileal portion of the small intestine.

In some embodiments, the microencapsulated IgY is prepared in a suspension for oral administration.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

The formulations provides by the present invention may be administrated to a subject prophylactically or after a presumed exposure to a pathogen, or after the onset of symptoms of TD.

In some embodiments, the formulation of the present invention comprising one or more therapeutically effective mixtures of anti-ETEC and anti-NV polyclonal IgY is administered prophylactically. That is, the formation may be administered prior to an anticipated possible exposure to either organism, i.e., prior to embarkation on a trip to a less-developed part of the world. In this aspect, the mixture is used as prophylaxis aimed at preventing infection or manifestations of disease by either microorganism, and is protective so long as the therapeutically effective amount of IgY is regularly administered. Use of IgY for prophylaxis against TD caused by either ETEC or HuNV is highly specific, and avoids impact on other microorganisms in the gut (the healthy gut microbiome). This is an advantage over use of antibiotics, which are known to disrupt normal microbiota.

Further advantages of the current invention include the ability to rapidly alter the composition of the prophylactic mixture in a timely fashion after the appearance of new variants of either microorganism.

In some aspects, prophylactic administration of the one or more formulations of the present invention is preferred over use of vaccines because of the immediate action of oral IgY, once delivered to the intestinal site of action. Vaccines require a period of days to weeks to achieve the full immune-modulating effect on the host, which is a disadvantage for short-term travelers and those with unexpected travel plans.

A related advantage of this invention over vaccination, in addition to its immediate onset, is its rapid offset of action within a short period of discontinuation of administration. This is an advantage particularly for the growing number of people who are vaccine “hesitant,” in that it avoids any permanent change in the individual's immune system.

A further advantage of this invention's IgY use as TD prophylaxis is that, unlike vaccines and other passive immunoprophylactics, the IgY specified in this invention provides coverage of both the leading bacterial strains and the leading viral strains causative of TD. This broader coverage is advantageous to the traveler, who will not have a priori knowledge of the specific pathogens likely to be encountered.

In another embodiment, the formulation comprising one or more therapeutically effective mixtures of anti-ETEC and anti-NV polyclonal IgY is administered after a probable exposure with the expectation of preventing or minimizing risk of infection or manifestation of symptoms of AGE.

In yet another embodiment, the formulation comprising one or more therapeutically effective mixtures of anti-ETEC and anti-NV polyclonal IgY is administered after the onset of symptoms of AGE, with the expectation of mitigating symptom severity and, importantly, reducing shedding of either pathogenic microorganism that places other individuals at risk.

The present invention has additional advantages over two additional commonly-recommended means of avoiding TD:

Dietary avoidance, meaning the avoidance of foods prepared locally that are likely to transmit either ETEC or HuNV, is both burdensome for the traveler and widely recognized to be ineffective. Both organisms are known to be infectious at very low numbers of individual microorganisms, and dietary avoidance is simple impractical and ineffective against such organisms.

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The therapeutically effective amount or dose of a compound of the present invention depends on the age, sex and weight of the patient, the current medical condition of the patient and the progression of a disease or disorder contemplated in the invention.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Suitable doses of a compound of the present invention may vary across a wide range of values, depending on the degree of decrease or increase in the relative abundance of targeted microorganisms desired. The dose may be administered in a single dosage or in multiple dosages, for example from 1 to 4 or more times per day. When multiple dosages are used, the amount of each dosage may be the same or different. For example, a dose of 1 g per day may be administered as two 0.5 g doses, with about a 12-hour interval between doses.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. The frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED₅₀. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

### Oral Administration:

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Additional Administration Forms:

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 min up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 min, about 20 min, or about 10 min and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 min, about 20 min, or about 10 min, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application. The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Production of Anti-ETEC Adhesin-tip MEFA IgY IgY Targets

To overcome problems posed by genotypic and phenotypic variance amongst pathogenic strains of ETEC, an adhesin-tip multi-epitope fusion antigen (MEFA) representing common ETEC adhesins was used for anti-ETEC IgY preparation. Adhesins and ETEC isolates from which they were derived are shown in Table 1.

TABLE 1
Enterotoxigenic Escherichia coli strains used in design
of ETEC adhesion-tip MEFA.

| Strains | Relevant properties |
|---|---|
| H10407 | O78:H11; CFA/I, LT, STa |
| THK38/pEU405 | CS1 |
| DH5α/pEU588 | CS2 |
| E116 (E19446) | CS3, LT, STa |
| E106 (E11881/9) | CS4/CS6, LT, STa |
| UM 75688 | CS5/CS6, LT, STa |
| 2423 ETP98066 | CS6, LT, STa |
| JF2101 | CS21/EtpA/EatA, STa |
| JF2318 ETP050008 | EtpA, STa |

ETEC strains used to determine impact of anti-MEFA IgY on MEFA-design strains.

### Immunization of Laying Hens

Eight Commercial White Rock and Rhode Island cross-bred, sexlink hens (Pinola Hatchery, Shippensburg, Pa., USA) were housed in a purpose-built henhouse permitting segregation of paired hens. Hens were acclimated for two weeks prior to immunization at ambient temperatures on a 12-hour light/dark cycle on ad libitum water and a commercial diet (Martin's Layer Mash 16%, Martin's Elevator, Inc., Hagerstown, Md., USA). Protocols for hen maintenance and immunization were approved by the Scaled Microbiomics, LLC Animal Use and Care Committee (approval number 19-01-TD).

Adhesin-tip MEFA protein was added to avian adjuvant Montanide ISA 70 VG (Seppic, Inc., La Garenne-Colombes, France) and phosphate-buffered saline (PBS) mixture (7:3 v/v) in a high-shear blender to a final concentration of 100 μg of MEFA protein, and filter sterilized using a 0.2 μm pore size polyethersulfone filter membrane (VWR International, Radnor, Pa., USA). Sterility was confirmed by absence of visual growth after inoculating 25 μl of each vaccine mixture into fastidious BBLTM Schaedler broth with Vitamin K₁ (Becton Dickinson, Sparks, Md., USA) and incubating for 48 without aeration at 37° C. On day 1 of hen immunizations, four hens were injected with 0.5 ml in each breast muscle, delivering a total of 100 μg of ETEC adhesin-tip MEFA per hen. Booster injections were administered in an identical fashion on days 14 and 28 of hen immunizations. Two additional hens, designated “sham injected”, received 0.5 ml of PBS and adjuvant only, prepared as previously described without MEFA protein, in each breast during immunizations (days 1, 14, and 28). A final hen pair was used for control and received no immunizations, designated “unimmunized.”

### IgY Extraction and Concentration

Two eggs were collected weekly from each hen pair beginning one day prior to the first immunization injections. IgY was extracted from yolks using polyethylene glycol (PEG), with the following modifications. Briefly, yolks were pooled, and lipid content was removed by centrifugation (13,000×g for 20 min at 4° C.) using PEG 6000 at consecutively increasing concentrations (3.5, 8.5, and 12% w/v; Alfa Aesar, Haverhill, Mass., USA). The resulting precipitate was resuspended in PBS and dialyzed against sodium chloride (0.1% w/v) for 16 hours and PBS for an additional three hours using Spectra/Por 4 standard RC dialysis tubing (12-14 kD; Spectrum Laboratories, Inc, Rancho Dominguez, Calif.). The resulting water-soluble fraction (WSF) containing IgY was stored at −20° C. until further analysis (<two weeks). Total protein concentrations of WSF were determined by bicinchoninic acid (BCA) method kit (Thermo Fisher Scientific, Rockford, Ill., USA), following manufacturer's specifications. Absorbance values were read at 490 nm using THERMOmax microplate reader (Molecular Devices, Sunnyvale, Calif., USA), and standard curve showed linear behavior (R²=0.99) over seven serial 1:2 dilutions (0.06-2 mg/ml) of the bovine serum albumin protein standard set (Thermo Fisher Scientific, Waltham, Mass., USA).

Sodium Dodecyl sulfate-polyacrylamide Gel Electrophoresis (SDS-PAGE)

To determine purity of yolk-derived IgY, SDS-PAGE was conducted under reducing conditions using 12% polyacrylamide gel (NuSep Inc., Germantown, Md., USA) with a Novex Mini-Cell (Invitrogen, Carlsbad, Calif., USA). Briefly, purified WSF samples were diluted 1:10 in PBS, mixed with equal volume of sample buffer, and denatured for 5 min at 100° C. A total of 20 μ1 of sample/buffer mixture was loaded into each well, and protein bands were visualized with Protein Fixative (Ward's Science, Rochester, N.Y., USA), as recommended by the manufacturer. Gels were imaged using a standard camera.

### Enzyme-Linked Immunosorbent Assay: IgY in MEFA Immunized Hens' Eggs

IgY titers against the adhesin-tip MEFA were measured by indirect noncompetitive ELISA. Briefly, 96-well flat bottom microtiter plates were coated with 400 ng of MEFA, after which plates were blocked with 5% nonfat milk and incubated with three serial 1:2 dilutions of IgY for one hour at room temperature (23-25° C.). Bound anti-MEFA IgY was detected by horseradish peroxidase (HRP)-conjugated goat anti-chicken IgY (1:2,500; ImmunoReagents, Inc., Raleigh, N.C., USA). Plates were washed five times using commercial ELISA wash buffer (Thermo Fisher Scientific, Waltham, Mass., USA) and visualized using 3,3′-5,5′-tetramethylbenzidine (TMB; VWR International, Radnor, Pa., USA). Optical density (OD) was measured on a THERMOmax microplate reader (Molecular Devices, Sunnyvale, Calif., USA) at 450 nm. Antigen-specific IgY titer was defined as the maximum dilution multiple of the sample with an OD value that was 2.1 times the unimmunized control.

### Adhesin-tip MEFA Is A Strong Immunogen in Avians

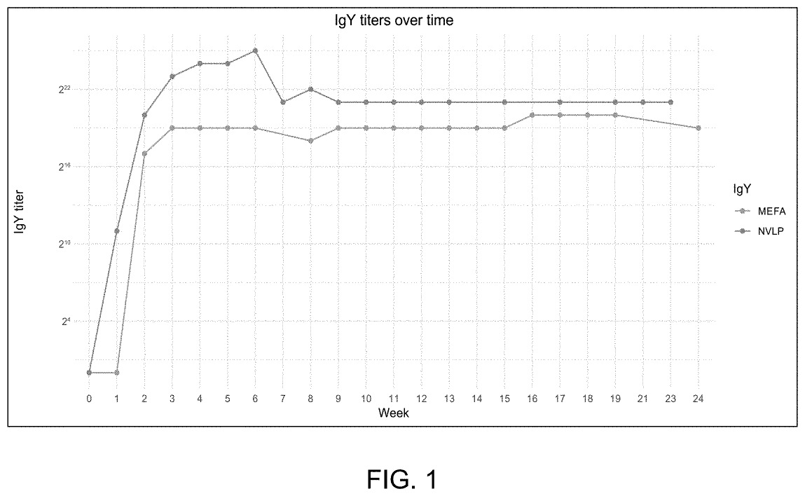

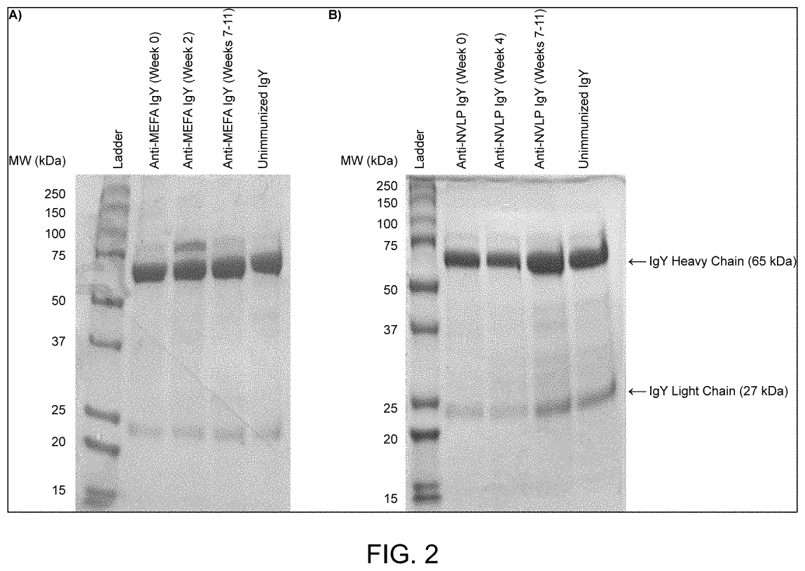

ELISA of IgY production in hens immunized with adhesin-tip MEFA showed antibody production by three weeks post-immunization, achieving titers of 1:524,288 (2¹⁹) at 9 weeks. Furthermore, production of anti-adhesin-tip MEFA IgY was sustained at or above these titers until at least 23 weeks, when the recording period ended (FIG. 1). By contrast, ELISA of both unimmunized and sham-immunized hens IgY revealed no detectable antigen-specific antibodies (not shown). Therefore, the control condition is hereafter referred to as “Unimmunized IgY.” SDS-PAGE revealed bands at the molecular weights expected for purified IgY, with a heavy chain at 68 kD and light chain 24 kD, respectively (FIG. 2). The concentration of purified IgY in PBS after dialysis was approximately 10 mg/ml, determined by BCA assay. This material was used in all subsequent analyses.

### Example 2

### Anti-ETEC Adhesin-Tip MEFA IgY Specifically Binds to All Adhesin Tip Epitopes

### Enzyme Linked Immunosorbent Assay: Individual CFAs

In order to determine IgY binding to individual adhesins represented on the adhesin-tip MEFA, anti-CFA adhesins and anti-EtpA IgY antibody titers were measured by ELISAs as previously described. Briefly, wells of 2, 96-well microtiter plates (Thermo Scientific, Rochester, N.Y., USA) were coated with 100 ng of each recombinant adhesin tip subunit protein including CfaE (CFA/I), CooD (CS1), CotD (CS2), CstH (CS3), CsaE (CS4), CsfD (CS5), CssB (CS6), LngA (CS21) and EtpA, respectively. After blocking with 10% nonfat milk, plates were incubated with 1:2 serially diluted chicken IgY samples, ranging from 1:200 to 1:51,200, at 37° C. for 1 hour and washed three times with PBS containing 0.05% Tween 20. Plates were incubated with HRP-conjugated goat anti-chicken IgY antibodies (1:10,000 dilution; Bethyl Laboratories, Montgomery, Tex.) at 37° C. for 1 hour. TMB Microwell Peroxidase Substrate System (Kirkegaard & Perry Lab Inc., Gaithersburg, Md.) was used to measure OD at 650 nm (0D650). IgY titers are presented as log transformation of the highest IgY sample dilution that produced an OD650 reading above 0.3 after subtraction of background. Initial titers were determined on one egg from each of two hens; upon demonstration that titers were similar between eggs, all subsequent studies were carried out with pooled IgY from multiple eggs.

### Immunized Hens Developed Immune Responses to All MEFA Adhesins

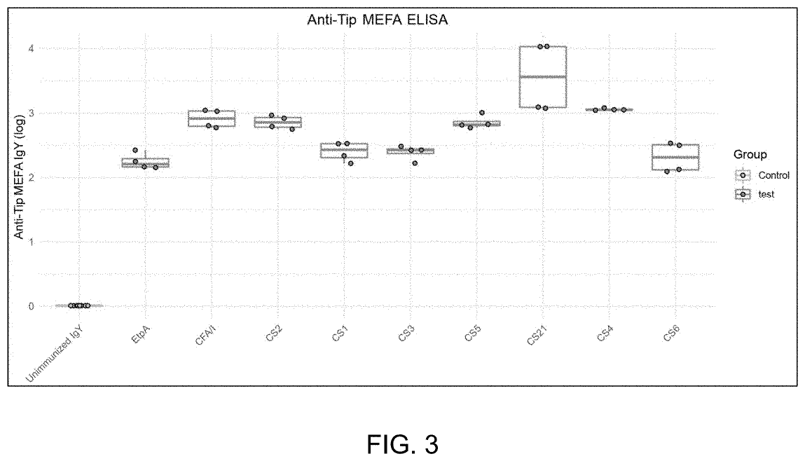

ELISA titers were detected for each of the nine MEFA adhesins following evaluation of anti-MEFA IgY (FIG. 3). In both hens, ELISA results showed anti-tip MEFA IgY (logio) titers of at least two against all adhesins. Unimmunized IgY did not show reactivity against any of the MEFA adhesins and resulted in non-detectable ELISA titers.

### Example 3

### Anti-ETEC Adhesin-Tip MEFA IgY Prevents ETEC Adhesion to Mammalian Cells

The requisite first step for all ETEC strains in establishing infection and elaborating the LT and ST toxins that induce diarrhea is successful adhesion to small intestinal epithelial cells. Prevention of that adhesion is a known means of preventing ETEC colonization and subsequent infection and toxin production.

### ETEC Adhesin-Tip MEFA-Targeted IgY: Antibody Adherence Inhibition Assays Impact of Anti-ETEC Adhesin-Tip-MEFA IgY on MEFA-design ETEC Strains

To evaluate anti-MEFA IgY neutralizing activities against bacterial adherence, we examined the ability of anti-MEFA IgY WSF to inhibit adherence to Caco-2 cells (HTB-37TM; American Type Culture Collection, Manassas, Va., USA) of ETEC strains used in design of the adhesin-tip MEFA (Table 1), as described previously. Briefly, ETEC isolates encoding CFA/I, CS1, CS2, CS3, CS4/CS6, CS5/CS6, CS6, CS21 or EtpA (Table 1) were grown in liquid culture to 106 CFU/ml and pre-treated with mannose (4% v/v). For each strain, three biological replicates were incubated with 15 μl of anti-MEFA or unimmunized IgY samples (10 mg IgY/m1 PBS) at 24° C. for 30 min with aeration. Each IgY sample/bacteria mixture was normalized to 300μl with Eagle's Minimum Essential Medium (American Type Culture Collection, Manassas, Va., USA) and added to 105 Caco-2 cells in a 48-well plate at a multiplicity of infection ratio of 10 bacterial cells per Caco-2 cell. After incubation in a 5% CO2 incubator at 37° C. for 1 hour, Caco-2 cells were rinsed with PBS and dislodged with 0.5% Triton X-100 (Sigma-Aldrich, St. Louis, Mo., USA). E. coli adherent to Caco-2 cells were collected, serially diluted (1:10) 3 times. Each dilution was spread on Luria-Bertani (LB) solid media plates (MP Biomedicals, Solon, Ohio, USA) and incubated overnight at 37° C. The following day, visible colonies were counted as colony forming units (CFU).

### Impact of Anti-ETEC Adhesin-Tip MEFA IgY on Clinical Outbreak ETEC Strains

To examine adherence-inhibition properties of IgY against eight ETEC strains expressing CFA/II, CFA/III, CFA/IV, or CS3 obtained clinically (Table 2), we employed methods similar to those used for MEFA-design strains, with slight modifications. All strains were prepared under standard growth conditions in LB broth at 37° C. with aeration overnight (16 hours) and normalized to an OD650 range of 0.6 using fresh LB broth. Bacteria were pre-treated with 4% (w/v) mannose and incubated with 45 μl of chicken IgY (10 mg/ml). After incubation at room temperature for 30 min with aeration, each IgY sample/bacteria mixture was normalized to 900 μl with PBS (40% v/v), and 180 μl of the bacteria/IgY mixture was added to 105 Vero cells (CCL-81™; American Type Culture Collection, Manassas, Va., USA) in each well of a 48-well microtiter plate. Vero cells were incubated and processed following the techniques described above to determine CFU count.

### Anti ETEC Adhesin-Tip MEFA IgY Inhibits ETEC Adherence to Mammalian Cells In Vitro

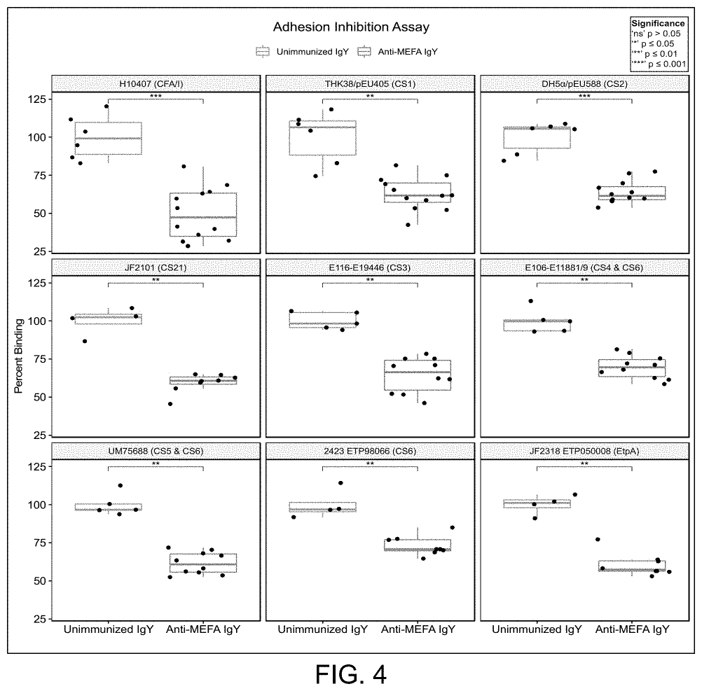

IgY extracted from pooled yolks of adhesin-tip MEFA-immunized hens inhibited adherence of ETEC strains to mammalian cells. FIG. 4 shows adherence-inhibition results for the nine strains used in the development of the MEFA (Table 1). Adherence of all MEFA-derivation ETEC strains examined in this study was inhibited by anti-MEFA IgY (P≤0.01). However, adherence inhibition varied by strain between about 25% (strain E106-E11881/9; encoding CS4/6) to 50% (strain H10407, encoding CFA/I) inhibition observed in anti-MEFA IgY compared to unimmunized IgY.

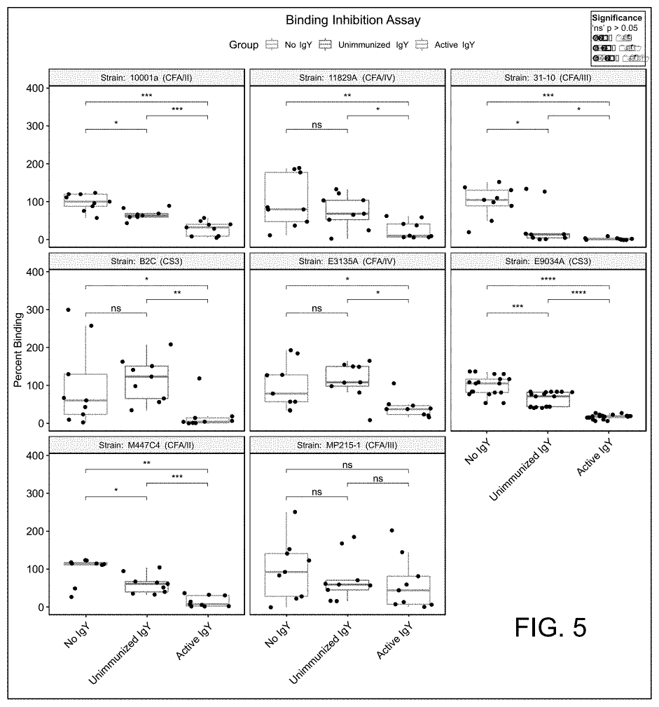

To determine if adhesin-tip MEFA IgY is capable of inhibiting adherence of ETEC strains different from those used in design of the MEFA, we also examined six isolates bearing CFA/CSs represented on the adhesin-tip MEFA, and 2 strains carrying CFA/III, which is not included on that MEFA (Table 2). Results demonstrated significant inhibition (P≤0.05) of adhesion for all strains except one of the CFA/III-encoding strains, MP215-1 (FIG. 5). We also included a “No IgY” condition to probe for general adhesion-inhibitory properties of non-specific IgY, and slight inhibition by unimmunized IgY compared with the “No IgY” condition was demonstrated in some strains.

TABLE 2
Enterotoxigenic Escherichia coli strains isolated from human patients
presenting with diarrhea

| Strains | Relevant properties |
|---|---|
| E9034A | CS3 |
| B2C | CS3 |
| E3135A | CFA/IV |
| 11829a | CFA/IV |
| 10001a | CFA/II |
| M447C4 | CFA/II |
| M633C1 | CFA/I |
| MP215-1* | CFA/III |
| 31-10* | CFA/III |

ETEC strains used to determine impact of MEFA on clinical outbreak ETEC strains.
“*” indicates strains encoding CFAs that are not represented on the ETEC adhesin tip MEFA.

### Example 4

### Anti-ETEC Adhesin-Tip MEFA IgY Has No Effect on E. coli Growth Rate

Because it is undesirable to have an anti-ETEC prophylactic impact the growth of either target or non-target bacteria, the IgY was evaluated to demonstrate any impact on growth of ETEC strains and non-adherent, commensal E. coli.

### Growth Inhibition Assays

Growth inhibition assays were conducted by OD method. Briefly, three biological replicates of ETEC isolates encoding CFA/II, CFA/III, CFA/IV, or CS3 (Table 1) and a commensal E. coli strain (BL21; Genotype: F-, ompT, hsdSB (rB-, mB-), dcm, gal, λ(DE3), pLysS, Cmr; Promega, Madison, Wis., USA), that does not encode any of the adhesins included on MEFA, were grown in LB broth overnight at 37° C. with aeration and normalized to an OD650 of 0.06 using fresh LB broth. Anti-MEFA IgY or unimmunized IgY (0.4 mg) in 40 μl of PBS was added to each well of a 96-well microtiter plate. PBS was used as a blank control. Ciprofloxacin hydrochloride in PBS (1 ng/μl) was used as negative assay control. A total of 260 μl of LB broth was added to each well and inoculated with 20 μl of each normalized culture, respectively. Cultures were incubated at 37° C. with aeration for up to 18 h, and OD650 was recorded hourly using a THERMOmax microplate reader (Molecular Devices, Sunnyvale, Calif., USA). Growth rates were presented as the maximum hourly change in OD650 for each isolate across three technical replicates.

### Target-Specific IgY Effects on Growth of Bacterial Strains

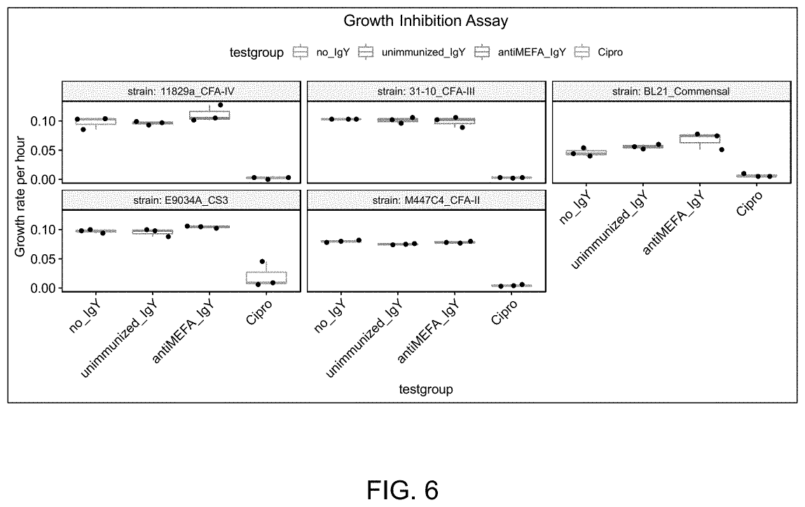

Growth of all isolates was inhibited by ciprofloxacin hydrochloride, as expected; however, there was no detectable effect of anti-ETEC adhesin-tip MEFA IgY on bacterial growth for any of the eight clinical isolates or of the commensal strain examined under the growth conditions included here (FIG. 6).

### Example 5

### Production of anti-HuNV GII.4 CHDC2094/1974 IgY

IgY targets

HuNV-like particles (HuNVLP) representing Norovirus GII.4, the predominant NV genotype in outbreaks of gastroenteritis globally,{Hallowell, 2019 #3245; Netzler, 2019 #3246} were obtained from The Native Antigen Company (Oxfordshire, United Kingdom). HuNVLP used in this study were structured from the genome of HuNVGII.4/CHDC2094/1974/US.

### Immunization of Laying Hens

Eight Commercial White Rock and Rhode Island cross-bred, sexlink hens (Pinola Hatchery, Shippensburg, PA, USA) were housed in a purpose-built hen-house permitting segregation of paired hens. Hens were acclimated for two weeks prior to immunization at ambient temperatures on a 12-hour light/dark cycle on ad libitum water and a commercial diet (Martin's Layer Mash 16%, Martin's Elevator, Inc., Hagerstown, Md., USA). Protocols for hen maintenance and immunization were approved by the Scaled Microbiomics, LLC Animal Use and Care Committee (approval number 19-01-TD).

HuNVLP were added to avian adjuvant Montanide ISA 70 VG (Seppic, Inc., La Garenne-Colombes, France) and phosphate-buffered saline (PBS) mixture (7:3 v/v) in a high-shear blender to a final concentration of 100 μg of HuNVLP per ml, and filter sterilized using a 0.2 μm pore size polyethersulfone filter membrane (VWR International, Radnor, PA, USA). Sterility was confirmed by absence of visual growth after inoculating 25 μl of each vaccine mixture into fastidious BBL™ Schaedler broth with Vitamin K₁ (Becton Dickinson, Sparks, Md., USA) and incubating for 48 without aeration at 37° C. On day 1 of hen immunizations, four hens were injected with 0.5 ml in each breast muscle, delivering a total of 100 μg of NVLP per hen. Booster injections were administered in an identical fashion on days 14 and 28 of hen immunizations. Two additional hens, designated “sham injected”, received 0.5 ml of PBS and adjuvant only, prepared as previously described without MEFA protein or NVLP, in each breast during immunizations (days 1, 14, and 28). A final hen pair was used for control and received no immunizations, designated “unimmunized.”

### IgY Extraction and Concentration

Two eggs were collected weekly from each hen pair beginning one day prior to the first immunization injections. IgY was extracted from yolks using polyethylene glycol (PEG). Briefly, yolks were pooled, and lipid content was removed by centrifugation (13,000×g for 20 min at 4° C.) using PEG 6000 at consecutively increasing concentrations (3.5, 8.5, and 12% w/v; Alfa Aesar, Haverhill, Mass., USA). The resulting precipitate was resuspended in PBS and dialyzed against sodium chloride (0.1% w/v) for 16 hours and PBS for an additional three hours using Spectra/Por 4 standard RC dialysis tubing (12-14 kD; Spectrum Laboratories, Inc, Rancho Dominguez, Calif.). The resulting water-soluble fraction (WSF) containing IgY was stored at -20° C. until further analysis (<two weeks).

Total protein concentrations of WSF were determined by bicinchoninic acid (BCA) method kit (Thermo Fisher Scientific, Rockford, Ill., USA), following manufacturer's specifications. Absorbance values were read at 490 nm using THERMOmax microplate reader (Molecular Devices, Sunnyvale, Calif., USA), and standard curve showed linear behavior (R²=0.99) over seven serial 1:2 dilutions (0.06-2 mg/ml) of the bovine serum albumin protein standard set (Thermo Fisher Scientific, Waltham, Mass., USA).

Sodium Dodecyl sulfate-polyacrylamide Gel Electrophoresis (SDS-PAGE)

To determine purity of yolk-derived IgY, SDS-PAGE was conducted under reducing conditions using 12% polyacrylamide gel (NuSep Inc., Germantown, Md., USA) with a Novex Mini-Cell (Invitrogen, Carlsbad, Calif., USA). Briefly, purified WSF samples were diluted 1:10 in PBS, mixed with equal volume of sample buffer, and denatured for 5 min at 100° C. A total of 20 μl of sample/buffer mixture was loaded into each well, and protein bands were visualized with Protein Fixative (Ward's Science, Rochester, N.Y., USA), as recommended by the manufacturer. Gels were imaged using a standard camera (FIG. 2).

### HuNVLPs are Strong Immunogens in Avians

ELISA of IgY production in hens immunized with HuNVLP showed detectable antibody production by three weeks after the first immunization, achieving post-immunization titers of 1:2,097,152 (2²¹) at 9 weeks. IgY production of anti-HuNVLP was sustained at or above these titers until at least 23 weeks, when the recording period ended (FIG. 1). Recent sampling has demonstrated that at nine months post-immunization titers remain at least 1:131,072 (2¹⁷). By contrast, ELISA of both unimmunized and sham-immunized hens IgY revealed no detectable antigen-specific antibodies (not shown). Therefore, the control condition is hereafter referred to as “Unimmunized IgY.”

SDS-PAGE revealed bands at the molecular weights expected for purified IgY, with a heavy chain at 68 kD and light chain 24 kD, respectively (FIG. 2). The concentration of purified IgY in PBS after dialysis was approximately 10 mg/ml, determined by BCA assay. This material was used in all subsequent analyses.

### Example 6

### Anti-GII.4 CHDC2094/1974 IgY Prevents HuNV VLP Adhesion to HBGA

### HuNVLP-Targeted IgY: Adherence Inhibition Assays

Pig gastric mucin (PGM), Type III with HBGA type A, Ley and H2, (PGM; Sigma Aldrich, St. Louis, MO, USA) was used in an antibody-blocking assay. Briefly, PGM was resuspended in PBS, and 1μg was coated onto 96-well U-bottom vinyl microtiter plates (Thermo Fisher Scientific, Rockford, Ill., USA) by adding 100 μl/well for 4 hours at room temperature. Plates were blocked overnight at 4° C. in 5% skim milk in 0.05% Tween 20-PBS. GII.4 NVLP's were pre-treated for 1 hour at room temperature with decreasing concentrations of anti-NVLP IgY, beginning with a starting concentration of 4 ng/l and followed by five serial tenfold dilutions. A total of 100 μl of the HuNVLP-IgY mixture was transferred to the PGM coated plates and incubated for 1 h at 37° C. Plates were washed three times with 0.05% Tween 20-PBS, and bound HuNVLPs were detected using a diluted (1:10,000) monoclonal anti-GII.4 VP1 VLP, mouse IgG (LifeSpan BioSciences Inc., Seattle, Wash., USA), following incubation for 1 h at 37° C. Plates were washed again as mentioned and incubated with anti-mouse, goat IgG-HRP conjugated antibodies (Azure Biosystems, Dublin, Calif., USA) at a 1:2,000 dilution for lh at 37° C. Following a final series of five washes, the assay was developed with commercial TMB substrate (VWR International, Radnor, PA, USA) using 100 μl/well. The OD650 was measured using THERMOmax microplate reader (Molecular Devices, Sunnyvale, Calif., USA), every 5 minutes, for up to 40 min until linearity was established. Binding inhibition was expressed as percent absorbance of the uninhibited blank control and confirmed by comparison to unimmunized control IgY.

### Anti-HuNVLP IgY Inhibits VLP Adhesion to Histo-Blood Group Antigens In Vitro

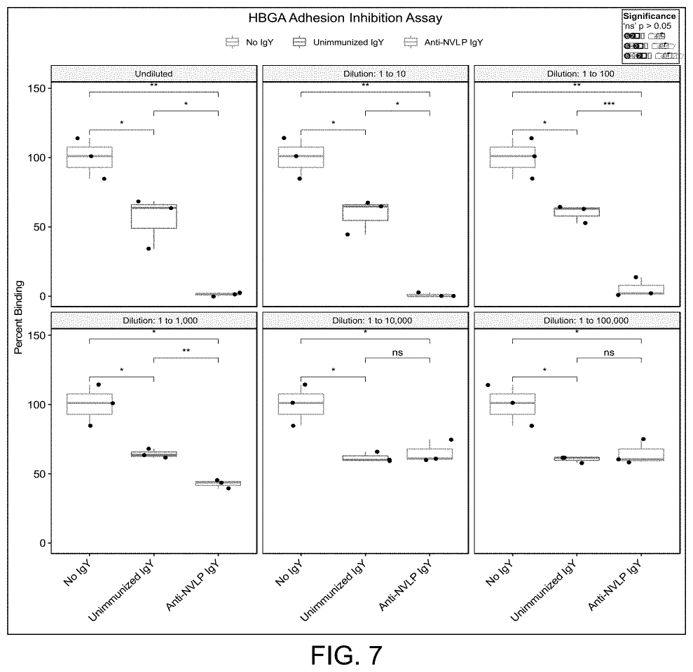

In an in vitro cell-free system using HBGA as the adhesion target, anti-GII.4 CHDC2094/1974 IgY significantly inhibited (P≤0.01) binding of GII.4 CHDC2094/1974 VLP to the adhesion target compared with both the No IgY and Unimmunized IgY conditions at dilutions from “neat” (10 mg protein/ml PBS) to 1:1000 (FIG. 7). Dose dependency was apparent up to 1:1000, while Unimmunized IgY demonstrated a significant inhibition of adhesion compared with No IgY at these concentrations, which was significantly less than that produced by the active IgY. At dilutions of 1:10,000 and 1:100,000, we observed no difference in adhesion inhibition between the Unimmunized IgY and anti-NVLP IgY.

### Example 7

### HuNV Neutralization in Human Intestinal Enteroids

To evaluate the impact of the anti-GII.4 CHDC2094/1974 IgY against live GII.4 HuNV, we used a human intestinal enteroid (HIE) model carried out at Vaccine and Infectious Disease Organization (VIDO) under support by the National Institute for Allergy and Infectious Diseases (NIAID; Contract No. HHSN-2722017-00038I). The live virus was a more recently-emerged HuNV strain, GII.4[P16] Sydney, a variant with known capsid sequence heterology from the immunizing 1974 strain.

Three-dimensional HIE J2 type cells were seeded onto collagen IV-coated 96-well plates at 9×10⁴ cells/well. Cells were grown on Proliferation-Intesticult medium (Stem Cell Technology) with 10 μtM Y-27632 dihydrochloride at 37° C., 5% CO₂ for 24 h. Cells were then grown on Differentiation-Intesticult medium for 4-5 days to two-dimensional confluent monolayers for HuNV infection.

### Neutralization Assays

A first range-finding experiment used IgY at 10-fold dilutions, “neat” through 1:1000. IgY exhibiting signs of inhibition of HuNV infection were further tested at 2-fold dilutions, “neat” through 1:2048. Dilutions were made in complete media without growth factors (CMGf-) supplemented with 500 μtM glycochenodeoxycholic acid (GCDCA) media. The 10% HuNV stool filtrates contained approximately 1×10⁷genome equivalent per mL. The stool filtrate was diluted 1000-fold in CMGf- with 500 μM GCDCA to a virus concentration of 1×10⁴ genome equivalent per ml.

### GII4 Sydney Neutralization by Anti-GII4 CHDC2094/1974 IgY

Equal volumes of diluted HuNV stool filtrates were mixed with the IgY solutions. The IgY-virus mixture was incubated at 37° C. in 5% CO2 for 2 hours. Then 100 μl of mixture was added to the pre-prepared 2D HIE cell monolayer. Cells with the IgY-virus mixture were incubated 1 hour at 37° C. in 5% CO_{2.} The mixture was then removed and cells were washed 3 times with CMGF-medium. Cells were incubated for another 24 hour in fresh media before harvesting for viral analysis. The infection experiment was performed in triplicate. Medium alone and medium with virus was used for controls of cell growth and infections. The viral polymerase inhibitor 2′-C-methylcytidine (2CMC) was used as a positive control.

After incubation, cells were harvested and RNA was extracted. Quantitative reverse transcriptase reactions were performed on the RNA samples. Primers were synthesized by Integrated DNA Technologies (IDT, Table 3). Dilutions of VIDO in-house HOV36 RNA transcript was used to generate a standard curve. The genome equivalents per well was obtained by comparing to the standard curve. A 5-FAM fluorescein probe and ZEN' fluorescence quencher were used with the probe.

TABLE 3
Primers Used in HuNV Viral Neutralization Study
Primers used in HuNV neutralization study

| | Sequence | |
|---|---|---|
| Description | Name | Sequence |
| TaqMan | QNIFS | /56-FAM/AGC ACG TGG/ |
| Probe | | ZEN/GAG GGC GAT CG/ |
| | | 3IABkFQ/ (SEQ ID NO: 1) |
| Forward | QNIF2d | ATG TTC AGR TGG ATG |
| Primer | | AGR TTC TCW GA |
| | | (SEQ ID NO: 2) |
| Reverse | COG2R | TCG ACG CCA TCT TCA |
| Primer | | TTC ACA (SEQ ID NO: 3) |

### Anti-GII.4 CHDC2094/1974 IgY Neutralizes Live GII.4[P16] Sydney HuNV in HIE

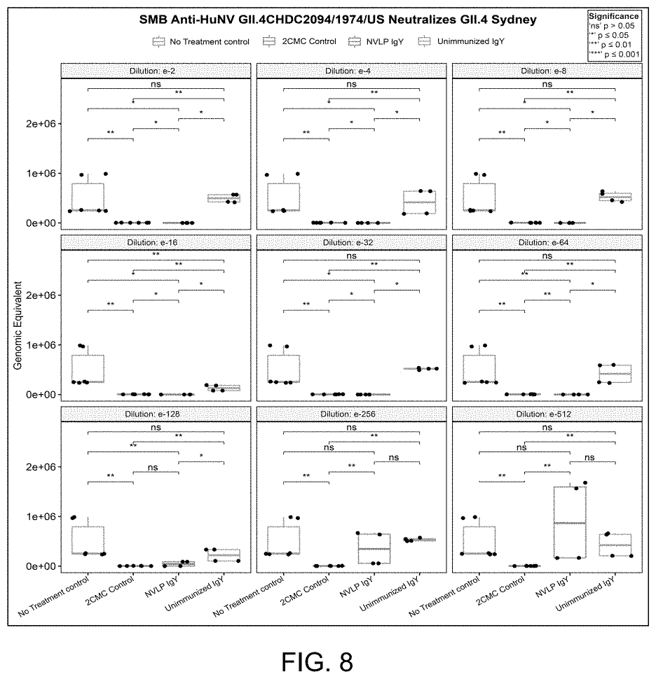

In the HIE model, anti-GII.4 CHDC2094/1974 VLP IgY demonstrated significant neutralization of live GII.4[P16] Sydney HuNV at dilutions from 1:2 to 1:128, compared with the No IgY and Unimmunized IgY conditions (FIG. 8). Neutralization by IgY was significantly greater than that produced by the small-molecule viral polymerase inhibitor 2CMC up to dilutions of 1:64 as well.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A formulation of antibodies containing both a) antibodies against multiple adhesin molecules of enterotoxigenic E. coli (ETEC) and b) antibodies against the VP1 capsid protein of Human Norovirus (NV).

2. The formulation of claim 1, wherein the antibodies comprise IgY antibodies.

3. The formulation of claim 1, wherein the molecule produced by an ETEC is an adhesin.

4. The formulation of claims 1, wherein the antibody binds a multiepitope fusion antigen (MEFA).

5. The formulation of claim 1, wherein the antibody inhibits ETEC adhesion to Caco-2 cells and to Vero cells.

6. The formulation of claim 1, wherein the formulation comprises a polyclonal mixture of antibodies.

7. The formulation of claim 1, wherein the antibodies against the VP1 capsid protein of Human Norovirus comprise an antibody that specifically binds a norovirus (NV) or epitope thereof

8. The formulation of claim 7, wherein the antibody of claim 7, wherein the antibody is an IgY antibody.

9. The formulation of claim 7, wherein the NV is NV GII.4 or NV GI.1.

10. The antibody of claims 7, wherein the antibody blocks binding of NV to histo-blood group antigen (HBGA).

11. A mixture of antibodies, the mixture comprising (a) an antibody that specifically binds an enterotoxigenic Escherichia coli (ETEC) or a molecule produced by an ETEC and (b) an antibody that specifically binds a norovirus (NV) or epitope thereof

12. The mixture of claim 11, wherein the antibody that specifically binds an enterotoxigenic Escherichia coli (ETEC) or a molecule produced by an ETEC is an antibody according to claim s 1, and wherein the antibody that specifically binds a norovirus (NV) or epitope thereof is an antibody according to claim 7.

13. The mixture of claim 11, wherein the mixture is multivalent.

14. The mixture of claim 11, wherein the mixture comprises a formulation for treating or preventing Travelers' diarrhea (TD).

15. The formulation of claim 14, wherein the formulation is formulated for oral administration.

16. A method of treating or preventing Travelers' diarrhea in a subject in need thereof, the method comprising administering to the subject the formulation of claim 14.

17. The method of claim 17, wherein the subject is a human.
